(19) United States
(12) Patent Application Publication
(10) Pub. No.: US 2005/0003471 A1
(43) Pub. Date: Jan. 6, 2005

(54) Methods of detecting megakaryocytes

(76) Inventors: Fu-Sheng Wang, Lake Zurich, IL (US); Mark S. Dorfner, Wonder Lake, IL (US); Suma Peesapati, Mundelein, IL (US); Steve M. Burger, Lakemoor, IL (US); Kojiro Hirai, Kobe (JP); Yukio Hamaguchi, Akashi (JP)

Correspondence Address: Brinks Hofer Gilson & Lione;NBC Tower, NBC Tower, Suite 3600, P.O. Box 10395, Chicago, IL, 60610, US

(21) Appl. No.: 10/613,767

(22) Filed: Jul. 3, 2003

(51) Int. Cl. C12Q 1/02; G01N 1/30; G01N 33/48

(52) U.S. Cl. 435/40.5

(57) ABSTRACT

Methods of detecting megakaryocytes are described that include (a) providing a sample that contains a cell; (b) detecting a plurality of morphological information from the cell; (c) generating a scattergram from the plurality of morphological information; and (d) determining whether a population exists in a megakaryocyte region of the scattergram.

### BACKGROUND

The present invention relates to methods of detecting megakaryocytes in human samples, such as blood and bone marrow. More particularly, the present invention relates to methods of detecting megakaryocytes accurately and precisely using an automated hematology analyzer.

Clinically, human hematopoiesis (i.e., the formation and development of different types of blood cells) is studied by examining blood and bone marrow samples. The counting and differentiation of blood cells provides information useful in the diagnoses of various diseases. For example, red blood cell (RBC) count provides information regarding erythropoiesis and anemia. White blood cell (WBC) count provides information regarding myelopoiesis and infection. Platelet count provides information regarding thrombopoiesis and hemostasis.

In addition to information obtained from the analysis of peripheral blood, information useful for clinical diagnoses may be obtained from an examination of the bone marrow. For example, megakaryocyte count and staging may provide important clinical information related to the diagnosis of hemostasis diseases and, furthermore, may facilitate differentiation of the pathologies of clinical disorders.

Traditionally, megakaryocytes (i.e., a precursor to blood platelets) have been detected and counted under a microscope—a very time consuming and labor intensive approach. The megakaryocyte count is determined manually and expressed as the total number of megakaryocytes per microscope slide. In addition to the drawbacks inherent in performing a manual counting method, other problems also exist. For example, with some diseases, such as myelodysplasia and megakaryocyte leukemia, megakaryocytes may appear in the peripheral blood expressed with different morphologies. Such cells are often ignored or counted as other types of cells.

An alternative method of analysis involves flow cytometry and antibodies. However, this method is multi-step and requires a complicated protocol of immunological procedures, such as staining, red blood cell lysing, washing, and the like.

In short, a rapid, automatic, and straightforward method for detecting megakaryocytes in a sample is needed to overcome the drawbacks associated with conventional technologies.

### SUMMARY

The scope of the present invention is defined solely by the appended claims, and is not affected to any degree by the statements within this summary.

Briefly stated, a first method of detecting a megakaryocyte embodying features of the present invention includes: (a) providing a sample that contains a cell; (b) detecting a plurality of morphological information from the cell; (c) generating a scattergram from the plurality of morphological information; and (d) determining whether a population exists in a megakaryocyte region of the scattergram.

A second method of detecting a megakaryocyte embodying features of the present invention includes: (a) preparing an assay sample by combining a sample containing a cell with a reagent; (b) detecting a plurality of information from the cell, wherein the information is selected from the group consisting of cell size information, cell interior information, degree of cell staining information, and combinations thereof; (c) generating a scattergram by plotting the plurality of information; and (d) determining whether a population exists in a megakaryocyte region of the scattergram.

A third method of detecting a megakaryocyte embodying features of the present invention includes: (a) preparing an assay sample by combining a sample comprising a cell with a reagent comprising a fluorescent dye and a hemolytic agent; (b) detecting scattered light and fluorescent light emitted by the cell; (c) generating a scattergram by plotting the scattered light and the fluorescent light; and (d) determining whether a population exists in a megakaryocyte region of the scattergram.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flow chart of a first method of detecting megakaryocytes embodying features of the present invention, wherein the megakaryocytes are collected from a megakaryocyte leukemia patient.

FIG. 2 shows a Wright-Giemsa stained cell photograph of undifferentiated Dami cells cultured in RIPM 1640 and Fetal Bovine Serum, which may represent progenitor or earlier stage megakaryocytes in human bone marrow.

FIG. 3 shows scattergrams of undifferentiated Dami cells tested on a Sysmex XE-2100 automated hematology analyzer using (a) regular settings and (b) altered settings.

FIG. 4 shows Wright-Giemsa stained cell photographs of the cell morphology of (a) undifferentiated Dami cells and (b) differentiated Dami cells.

FIG. 5 shows scattergrams of (a) undifferentiated Dami cells and (b) differentiated Dami cells tested on an automated hematology analyzer, wherein the extra cell populations appear in different windows such as differential, Baso, NRBC, and RBC.

FIG. 6 shows a flow chart of a second method of detecting megakaryocytes embodying features of the present invention, wherein the megakaryocytes are obtained from purified CD34 positive cells that have been cultured with thrombopoietin.

FIG. 7 shows (a) Wright-Giemsa stained cell photograph of the cell morphology of purified CD34 positive hematopoietic cells and (b) a scattergram of the purified CD34 positive hematopoietic cells tested on an automated hematology analyzer.

FIG. 8 shows a scattergram of the purified CD34 positive hematopoietic cells tested on an automated hematology analyzer prior to culturing with thrombopoietin.

FIG. 9 shows (a) Wright-Giemsa stained cell photograph of the cell morphology of megakaryocytes induced from CD34 positive hematopoietic cells cultured with thrombopoietin and (b) a scattergram of these megakaryocytes tested on an automated hematology analyzer.

FIG. 10 shows a scattergram of the purified CD34 positive hematopoietic cells tested on an automated hematology analyzer after 8 days of culturing with thrombopoietin.

FIG. 11 shows a scattergram of normal bone marrow.

FIG. 12 shows a scattergram of normal bone marrow.

### DETAILED DESCRIPTION

Throughout this description and in the appended claims, the following definitions are to be understood:

The terms “detecting,” “detection,” “analyzing,” and “analysis” (e.g., “detecting megakaryocytes,” etc.) refer to any quantitative, semi-quantitative, or qualitative method for determining an analyte in general, and a megakaryocyte cell in particular. For example, a method that merely detects the presence or absence of a megakaryocyte in a sample lies within the scope of the present invention, as do methods that provide data as to the amount or concentration of the cells in the sample.

The phrase “megakaryocyte region of a scattergram” refers to an area of a scattergram in which a population of megakaryocytes may be visible. In accordance with the presently preferred embodiments of the invention, it is presently preferred that at least about 15% of the plot in a megakaryocyte region of a scattergram results from a population of megakaryocytes, more preferably at least about 25%, more preferably at least about 35%, more preferably at least about 45%, more preferably at least about 55%, more preferably at least about 65%, and more preferably at least about 75%.

The term “undifferentiated” refers to cells corresponding to progenitor or early stage megakaryocytes.

The term “differentiated” refers to mature or late stage megakaryocytes.

It has now been discovered that megakaryocytes in a sample may be identified easily and without the use of immunological techniques by (1) preparing a scattergram from data collected from the sample, and (2) examining the scattergram for the presence of a megakaryocyte population. It has further been discovered that the plot corresponding to megakaryocytes appears in a characteristic region of the scattergram. The scattergram itself may be generated from data collected with an automated hematology analyzer. For purposes of comparison, a flow cytometer may be used to collect data in parallel control experiments.

Automated hematology analyzers, such as the multiparameter analyzers XE2100 and SE-9000 sold by the Sysmex Corporation of Kobe, Japan, are presently preferred for use in accordance with the present invention. The settings of the automated hematology analyzer are optimized for the detection of megakaryocytes since the detection of megakaryocytes would normally lie outside the range of settings routinely used with automated hematology analyzers.

The methods described herein allow an automated hematology analyzer to be used to examine human samples, such as blood and bone marrow, that contain megakaryocytes, and to analyze the staging as well as cell characteristic changes. The methods in accordance with the present invention may also be integrated into an automated instrument designed for bone marrow automation or to observe the whole picture of hematopoiesis.

Thus, a first method of detecting a megakaryocyte embodying features of the present invention includes: (a) providing a sample that contains a cell; (b) detecting a plurality of morphological information from the cell; (c) generating a scattergram from the plurality of morphological information; and (d) determining whether a population exists in a megakaryocyte region of the scattergram.

Preferably, the method further includes preparing an assay sample by mixing the sample containing the cell with a reagent such as a fluorescent dye (e.g., polymethine dye). The procedures for preparing the sample and the type of reagents that may be used therewith are analogous to those typically used in connection with the Sysmex XE-2100 automated hematology analyzer and described in the Sysmex XE-2100 Operator's Manual (Revised January 2003; see in particular Chapter 7). The entire contents of this Operator's Manual are incorporated herein by reference, except that in the event of any inconsistent disclosure or definition from the present application, the disclosure or definition herein shall be deemed to prevail.

The detection methods embodying features of the present invention have been developed in accordance with two alternative sources of megakaryocytes: (1) megakaryocyte cells or cell lines obtained from a patient; and (2) stem or progenitor cells that have been cultured in vitro. The megakaryocyte cells and cell lines from megakaryocyte leukemia patients and/or the megakaryocytes induced from hematopoietic stem and progenitor cells with thrombopoietin are presently preferred for use in accordance with the present invention.

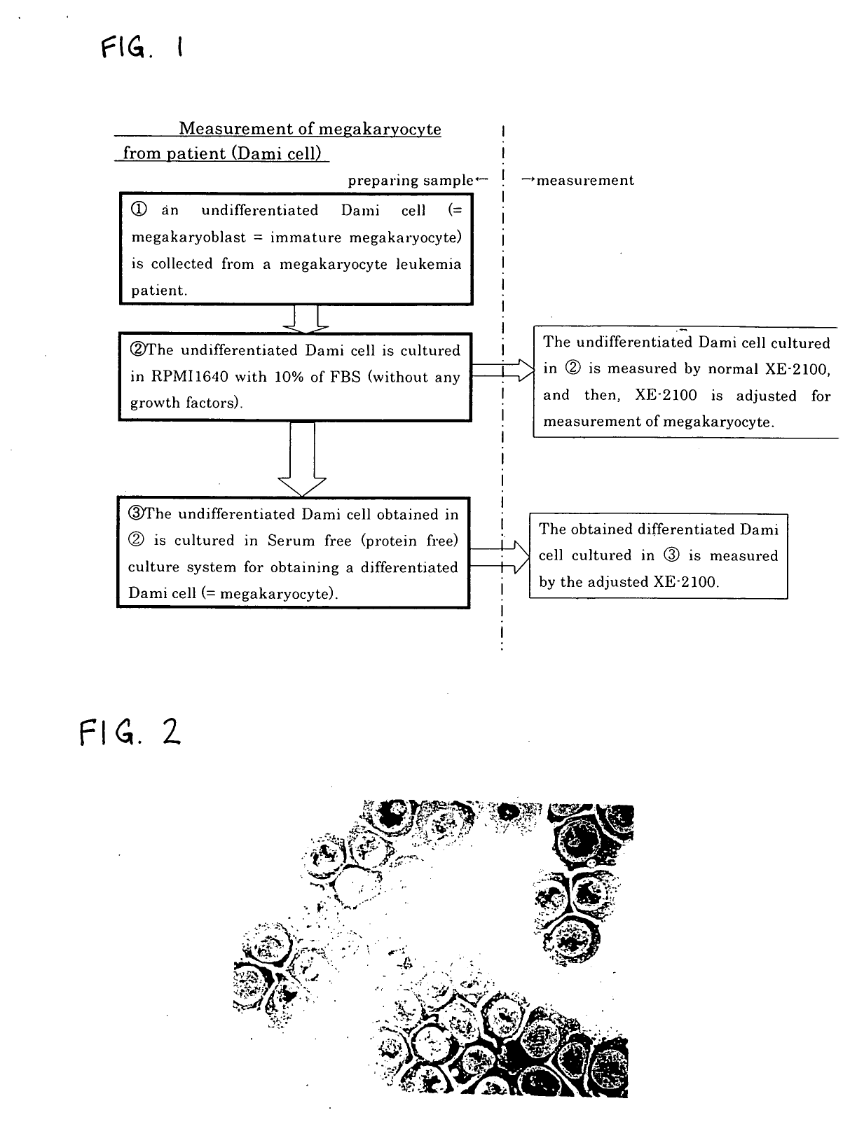

In accordance with the above-mentioned first source of megakaryocytes, Dami cells, a cell line obtained from a patient with megakaryocyte leukemia, are presently preferred megakaryocyte cells for use in accordance with the present invention. As outlined in the flow chart of FIG. 1, an undifferentiated Dami cell (i.e., a megakaryoblast or an immature megakaryocyte) is collected from a patient having megakaryocyte leukemia. The undifferentiated Dami cell is cultured in RPMI 1640 with 10% Fetal Bovine Serum (FBS) without any growth factor. FIG. 2 shows the morphology of undifferentiated Dami cells cultured in RIPM 1640 and Fetal Bovine Serum. The undifferentiated Dami cell thus cultured is measured on a Sysmex XE-2100 under normal settings shown in Table 1 below and under settings optimized for the measurement of megakaryocytes also shown in Table 1 below. This phase of testing enables the observation of cell behavior on the analyzer in order to establish a background for undifferentiated megakaryocytes on an automated hematology analyzer. Other megakaryocytes would behave similarly.

TABLE 1
Channel Numbers for Automated Hematology Analyzer

| PARAMETER | NORMAL SETTINGS | ADJUSTED SETTINGS |
|---|---|---|
| Diff-X | 1609 | 957 |
| Diff-Y | 474 | 295 |
| NRBC-X | 1762 | 0 |
| RBC-X | 400 | 1 |
| RBC-Y | 1637 | 824 |

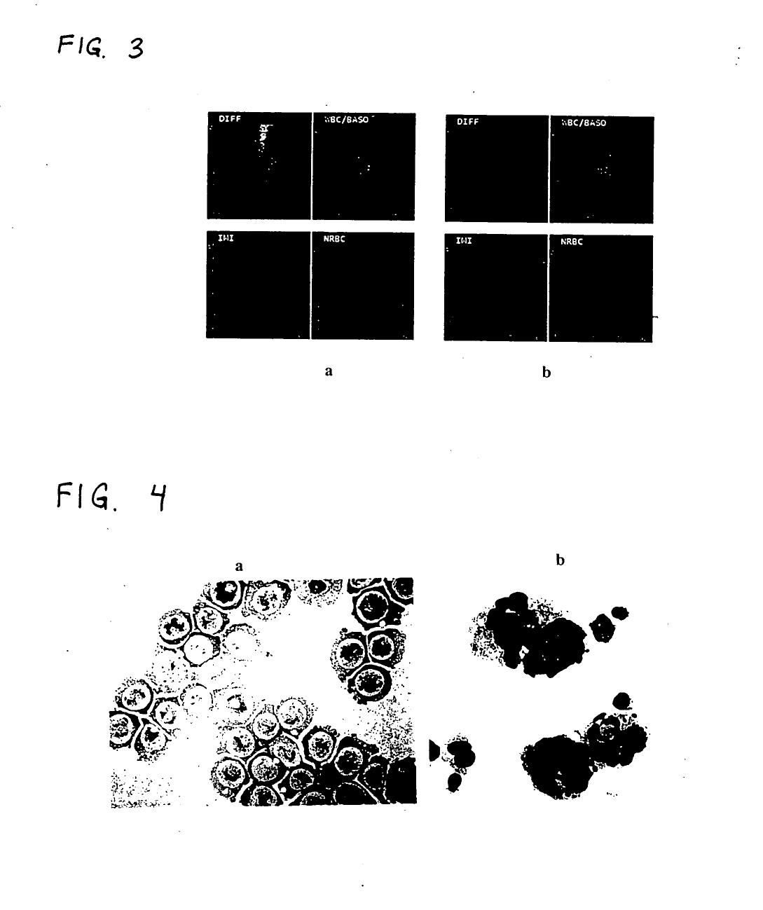

The optimized adjusted settings of the different windows were obtained using the undifferentiated Dami cells. The sensitivity of the parameters in the windows was reduced because the cell size and nucleic acid of Dami cells differ from that of normal peripheral blood cells. FIG. 3 shows scattergrams of the undifferentiated Dami cells tested using (a) the regular settings and (b) the adjusted settings.

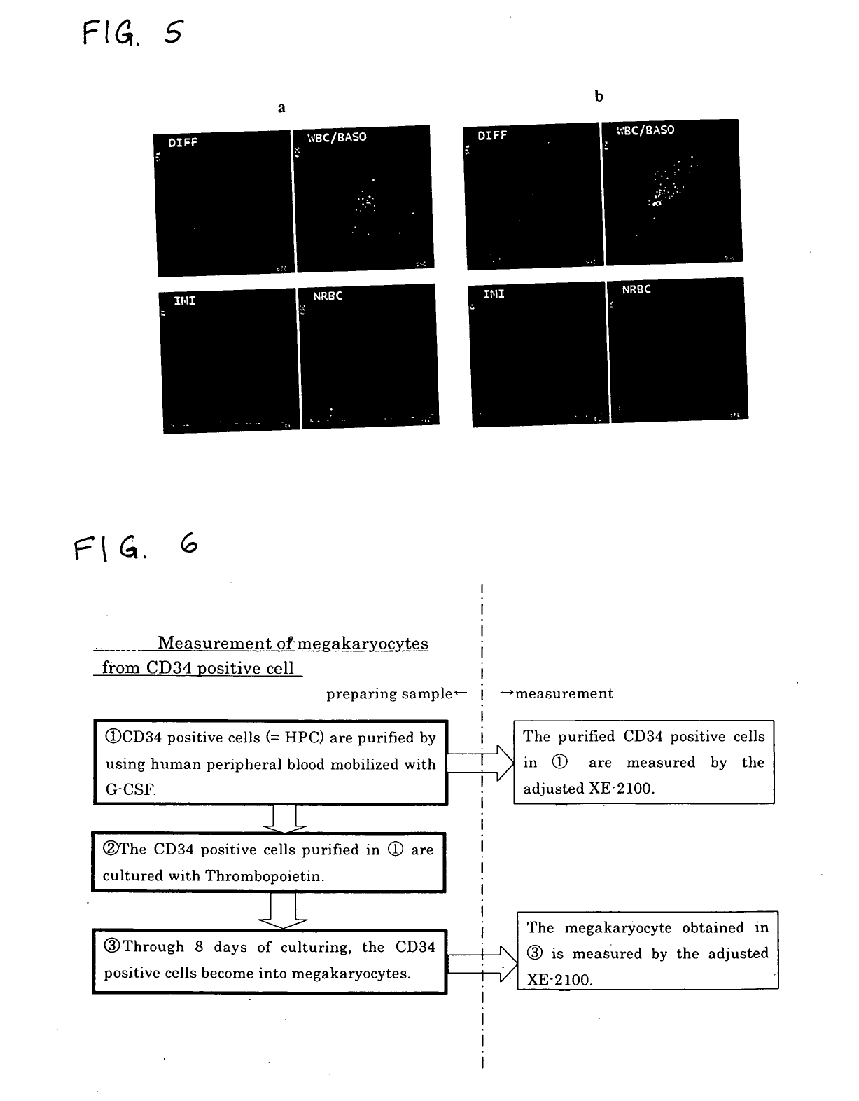

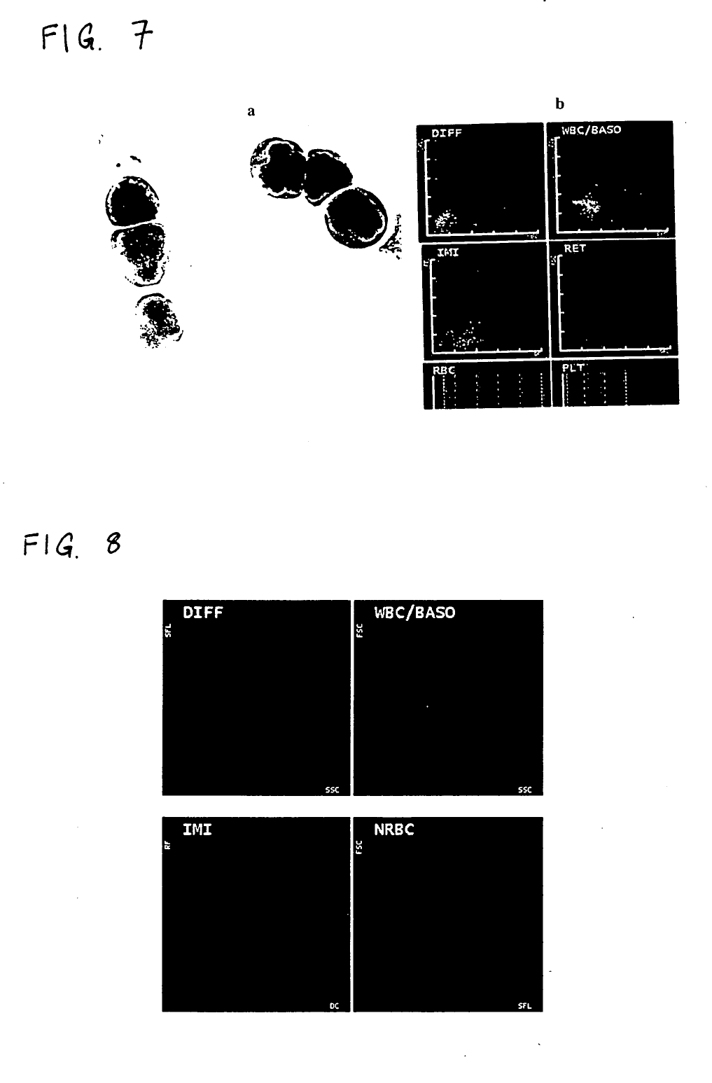

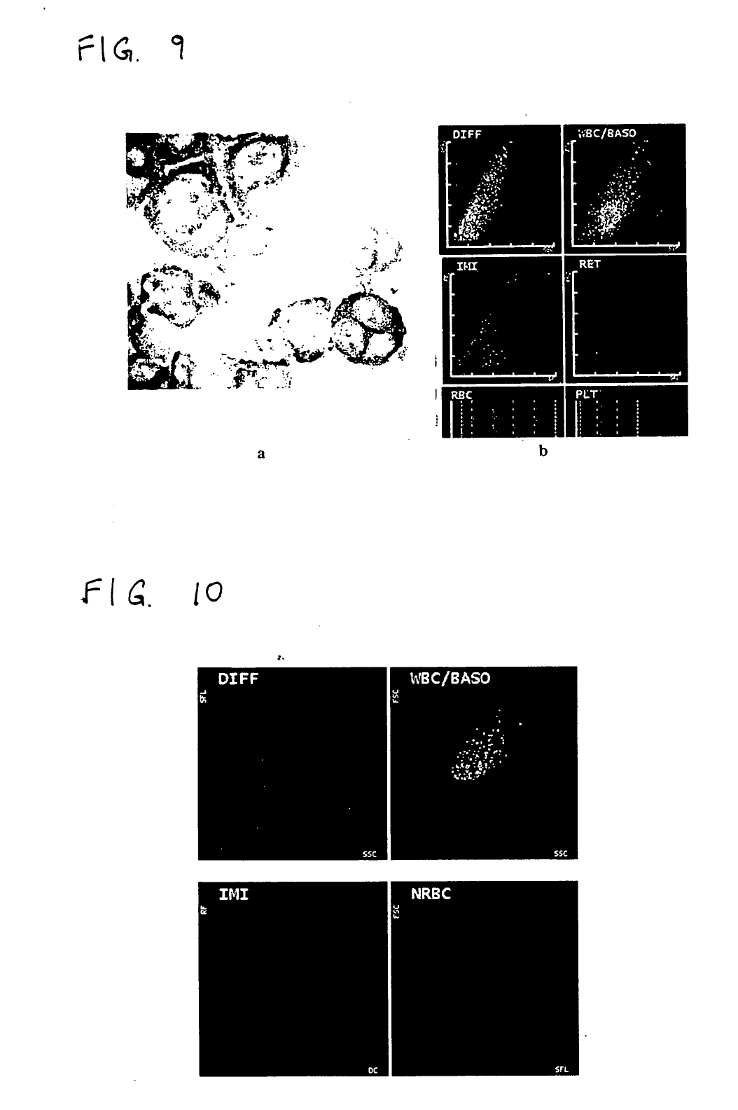

The undifferentiated Dami cell obtained above is then cultured in a serum free (i.e., protein free) cell culture system to obtain a differentiated Dami cell (i.e., a megakaryocyte). FIG. 4 shows the morphology of (a) undifferentiated Dami cells and (b) differentiated Dami cells. Cell morphology of the different Dami cells were observed with cytospin and Right-Giemsa staining that showed that the undifferentiated Dami cells are round with regular round nuclei and dark gray blue cytoplasm, whereas the differentiated Dami cells are approximately 2-5 times larger with irregular nuclei and rich pink cytoplasm, a morphology typical of mature or late stage megakaryocytes.

The differentiated Dami cells thus obtained are measured on a Sysmex XE-2100 using the adjusted settings shown in Table 1 above. The testing of the differentiated Dami cells on the automated hematology analyzer serves to facilitate determination of the cell population of mature/differentiated megakaryocytes in an automated hematology analyzer or similar cell analyzer. FIG. 5 shows scattergrams of (a) the undifferentiated Dami cells and (b) the differentiated Dami cells. The comparison study showed that the differentiated Dami cells had extra cell populations located in different scatter windows including the side scatter (SSC)/side fluorescent (SFL) in the differential window, the SSC/forward scatter (FSC) in the basophile window and the SFL/FSC in the NRBC (nucleated red blood cell) window and RBC window. The cell morphology showed that the undifferentiated cells were round with regular round nuclei, dark gray blue cytoplasm and small pseudopodia. The differentiated cells showed two to five-fold larger sizes with irregular shape of nuclei and rich pink cytoplasm, a typical mature morphology for megakaryocyte.

The Dami cell line described above typically has characteristics of an immature megakaryocyte. However, as noted, the cells can be treated under different conditions to become mature or differentiated megakaryocytes in cell morphology. In this study, the Dami cells were cultured in RPMI 1640 with 10% of Fetal Bovine Serum without any growth factor treatment for the undifferentiated cells and the mature/differentiated megakaryocytes were obtained in a protein free culture system. However, this induction can also be realized using different growth factors and under different conditions. Moreover, it should be emphasized that other megakaryocyte cell lines can be employed in place of Dami cells in accordance with the present invention.

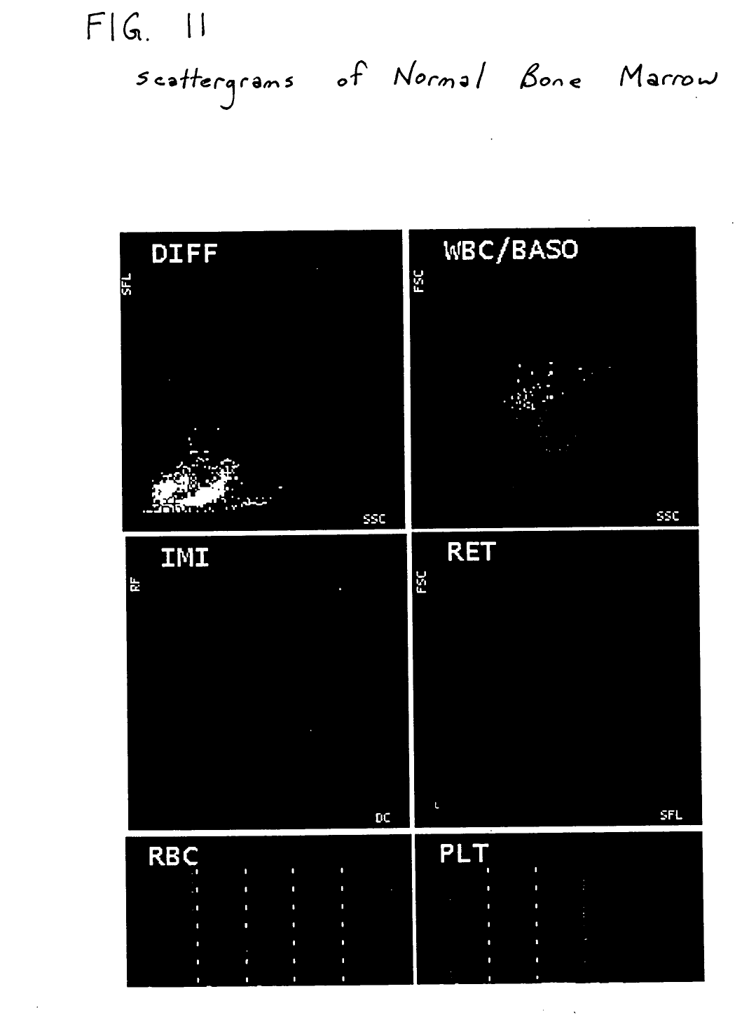

In accordance with the above-mentioned second source of megakaryocytes, hematopoietic cells cultured with thrombopoietin, and particularly purified CD34 positive cells cultured with thrombopoietin, are presently preferred for use in accordance with the present invention. As outlined in the flow chart of FIG. 6, CD34 positive cells (i.e., human hematopoietic progenitor cells, HPC) are purified using human peripheral blood mobilized with granulocyte colony stimulating factor (G-CSF). The purified CD34 positive cells represent an early stage of hematopoietic cells that can be induced to become different type of blood cells. FIG. 7(a) shows the morphology of purified CD34 positive hematopoietic cells. The purified CD34 positive cells thus obtained are then measured on a Sysmex XE-2100 using the adjusted settings shown in Table 1 above. FIG. 7(b) and FIG. 8 show scattergrams of the purified CD34 positive hematopoietic cells at day 1 (i.e., prior to culturing with thrombopoietin). The purified CD34 positive cells thus obtained are then cultured with thrombopoietin. After 8 days of culturing, the CD34 positive cells have been induced to become megakaryocytes having enlarged sizes, irregular nuclei, multi lobe nuclei and pink blue cytoplasm, as observed in authentic bone marrow samples. FIG. 9(a) shows the morphology of the megakaryocytes obtained from culturing CD34 positive hematopoietic cells. The megakaryocytes thus obtained are tested on the Sysmex XE-2100. All of the cells were studied freshly from the culture system on the instrument without pretreatment or staining. FIG. 9(b) and FIG. 10 show a scattergram of the megakaryocytes after 8 days of culturing.

As described above, differentiated Dami cells, undifferentiated Dami cells, CD34 positive hematopoietic cells, and megakaryocytes induced from CD34 positive hematopoietic cells may be analyzed on an automated hematology analyzer using the adjusted settings shown in Table 1. It should be emphasized that the settings themselves may vary with the type and/or condition of instrument used and that the numerical values shown in Table 1 are provided as guidelines.

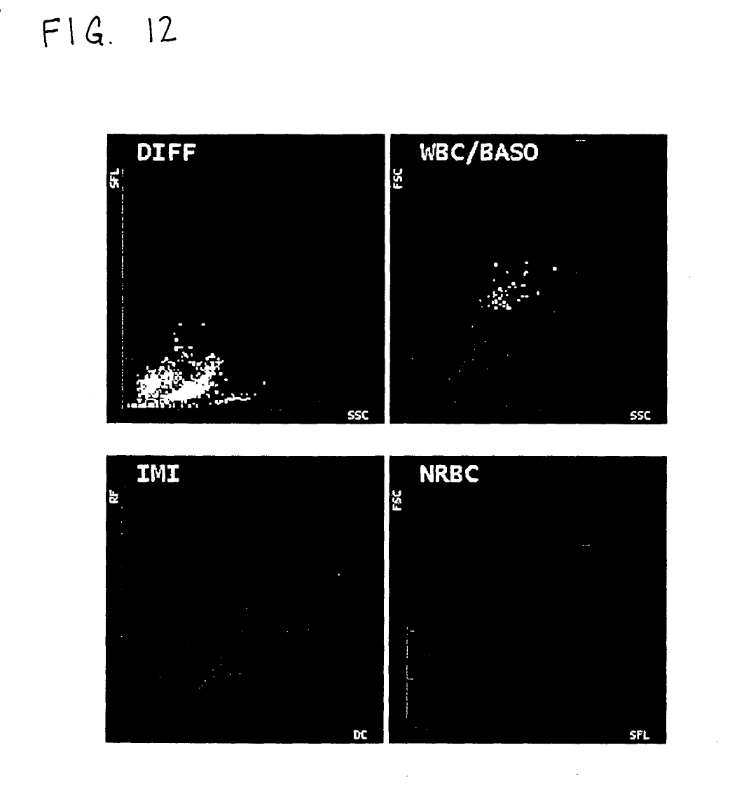

Identification of the characteristic megakaryocyte region of a scattergram involves a comparison of the scattergrams obtained from differentiated and undifferentiated Dami cells as well as the scattergrams of CD34 positive cells and megakaryocytes induced therefrom. For example, purified megakaryocytes are detected using a Sysmex XE-2100 with adjusted settings and a first scattergram is generated. A normal bone marrow sample is also examined using the Sysmex XE-2100 with adjusted settings and a second scattergram is generated. FIGS. 11 and 12 show scattergrams of normal bone marrow. By comparing the scattergram of the purified megakaryocytes with the scattergram of the normal bone marrow, a difference in distribution of cell population on the two scattergrams will be evident (e.g., especially in the high SFL area of the DIFF channel), which enables determination of the area in which only megakaryocytes appear. Thus, it is possible to locate the area occupied by cells with large and high fluorescent characteristics.

In a first series of preferred methods embodying features of the present invention, cells were tested on a Sysmex XE-2100 hematology analyzer and the megakaryocytes were observed in the differentiation channel. In these scattergrams, the x-axis represents side scatter (cell complexity) and the y-axis represents side fluorescence.

In a second series of preferred methods embodying features of the present invention, cells were tested on an XE-2100 hematology analyzer and the megakaryocytes were observed in the WBC/Baso channel. In these scattergrams, the x-axis represents the cell complexity and the y-axis represents cells size or forward scatter (FSC).

In a third series of preferred methods embodying features of the present invention, cells were tested on an XE-2100 hematology analyzer and the megakaryocytes were observed in the NRBC channel. In these scattergrams, the x-axis represents cell complexity or SFL and the y-axis represents cell size or forward scatter (FSC).

As is evident from the description above, megakaryocytes derived from either Dami cells or CD34 cells have characteristics typical of the cell lineage and can be used to detect megakaryocyte cell behavior on an automated hematology analyzer including but not limited to the Sysmex XE-2100.

Furthermore, it has been successfully demonstrated that an automated hematology analyzer such as the Sysmex XE-2100 may be used for the detection of megakaryocytes. This discovery will prove particularly useful in developing protocols for the observation of the whole picture of hematopoiesis or bone marrow automation with human specimens.

The foregoing detailed description and examples have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A method of detecting a megakaryocyte comprising:
(a) providing a sample comprising a cell;
(b) detecting a plurality of morphological information from the cell;
(c) generating a scattergram from the plurality of morphological information; and
(d) determining whether a population exists in a megakaryocyte region of the scattergram.

2. The invention of claim 1 further comprising preparing an assay sample by mixing the sample with a reagent.

3. The invention of claim 1 wherein the detecting involves an automated hematology analyzer.

4. The invention of claim 2 wherein the reagent comprises a fluorescent dye.

5. The invention of claim 2 wherein the preparing of the assay sample does not involve an immunological method.

6. The invention of claim 1 wherein the plurality of morphological information comprises side scattered light and fluorescent light emitted by the cell.

7. The invention of claim 1 wherein the plurality of morphological information comprises forward scattered light and side scattered light emitted by the cell.

8. The invention of claim 1 wherein the plurality of morphological information comprises forward scattered light and fluorescent light emitted by the cell.

9. The invention of claim 2 wherein the detecting comprises passing the assay sample through an electrically charged aperture and identifying a change in direct current resistance and radio frequency resistance.

10. The invention of claim 1 further comprising identifying the megakaryocyte region of the scattergram.

11. The invention of claim 10 wherein the identifying comprises:
generating a first reference scattergram from a plurality of information detected from a purified megakaryocyte;
generating a second reference scattergram from a plurality of information detected from a cell in at least one of a peripheral blood sample and a bone marrow sample, wherein the peripheral blood sample and the bone marrow sample are substantially free of megakaryocyte; and
comparing the first reference scattergram to the second reference scattergram, thereby identifying the megakaryocyte region.

12. The invention of claim 11 wherein the purified megakaryocyte comprises a differentiated Dami cell.

13. The invention of claim 11 wherein the purified megakaryocyte comprises a cell induced from a CD34 positive hematopoietic cell.

14. The invention of claim 13 wherein the cell is induced with thrombopoietin.

15. A method of detecting a megakaryocyte comprising:
(a) preparing an assay sample by combining a sample comprising a cell with a reagent;
(b) detecting a plurality of information from the cell, wherein the information is selected from the group consisting of cell size information, cell interior information, degree of cell staining information, and combinations thereof;
(c) generating a scattergram by plotting the plurality of information; and
(d) determining whether a population exists in a megakaryocyte region of the scattergram.

16. The invention of claim 15 wherein the detecting involves an automated hematology analyzer.

17. The invention of claim 15 wherein the reagent comprises a fluorescent dye.

18. The invention of claim 15 wherein the preparing of the assay sample does not involve an immunological method.

19. The invention of claim 15 wherein the cell interior information is detected based on side scattered light emitted by the cell, and the degree of cell staining information is detected base on fluorescent light emitted by the cell.

20. The invention of claim 15 wherein the cell size information is detected based on forward scattered light emitted by the cell, and the cell interior information is detected base on side scattered light emitted by the cell.

21. The invention of claim 15 wherein the cell size information is detected based on forward scattered light emitted by the cell, and the degree of cell staining information is detected base on fluorescent light emitted by the cell.

22. The invention of claim 15 wherein the detecting comprises passing the assay sample through an electrically charged aperture and identifying the cell size information based on a change in direct current resistance and the cell interior information based on a change in radio frequency resistance.

23. A method of detecting a megakaryocyte comprising:
(a) preparing an assay sample by combining a sample comprising a cell with a reagent comprising a fluorescent dye and a hemolytic agent;
(b) detecting scattered light and fluorescent light emitted by the cell;
(c) generating a scattergram by plotting the scattered light and the fluorescent light; and
(d) determining whether a population exists in a megakaryocyte region of the scattergram.

24. The invention of claim 23 wherein the scattered light comprises side scattered light.

25. The invention of claim 23 wherein the detecting involves an automated hematology analyzer.
